(19) United States
(12) Patent Application Publication
(10) Pub. No.: US 2023/0027857 A1
(43) Pub. Date: Jan. 26, 2023

(54) WASTE FLUID STORAGE CONTROL DEVICE

(71) Applicant: Murata Manufacturing Co., Ltd., Kyoto (JP)

(72) Inventors: Masaaki FUJISAKI, Kyoto (JP); Susumu TAKEUCHI, Kyoto (JP)

(21) Appl. No.: 17/937,591

(22) Filed: Oct. 3, 2022

Related U.S. Application Data
(63) Continuation of application No. PCT/JP2021/010497, filed on Mar. 16, 2021.

(30) Foreign Application Priority Data
Apr. 10, 2020 (JP) 2020-070693

(51) Int. Cl. G01M 3/32; G05D 16/20

(52) U.S. Cl. CPC G01M 3/32; G05D 16/2066

(57) ABSTRACT

A pump communicates with a waste fluid storage container and generates a negative pressure to draw waste fluids into the waste fluid storage container. A sensor measures a negative pressure. A driving unit performs driving control upon the pump. A computation unit detects, for example, the amount of air leak from the change in negative pressure. The driving unit performs driving control using an ON period Ton in which the pump operates and an OFF period Toff in which the pump stops. The computation unit detects the amount of air leak using the change in negative pressure in the OFF period Toff.

### CROSS REFERENCE TO RELATED APPLICATION

This is a continuation of International Application No. PCT/JP2021/010497 filed on Mar. 16, 2021 which claims priority from Japanese Patent Application No. 2020-070693 filed on Apr. 10, 2020. The contents of these applications are incorporated herein by reference in their entireties.

### BACKGROUND ART

### Technical Field

The present disclosure relates to a technique for detecting various states regarding drawing of waste fluids to be stored in a waste fluid storage device.

Patent Document 1 discloses an air leak monitoring device. The air leak monitoring device disclosed in Patent Document 1 includes a pump, a pressure sensor, and a control unit.

The pump draws air inside a waste fluid storage unit to cause a negative pressure. The pressure sensor measures the negative pressure of the waste fluid storage unit. The controller monitors an air leak using minimum negative pressure information in which the minimum negative pressure value of the waste fluid storage unit and a time are associated with each other.
Patent Document 1: Japanese Patent No. 6403545

### BRIEF SUMMARY

However, the air leak monitoring device disclosed in Patent Document 1 cannot accurately measure a negative pressure because the pulsation of the pump transmits to the pressure sensor. Accordingly, various states regarding drawing of waste fluids, such as an air leak, cannot be accurately detected.

The present disclosure accurately detects various states regarding drawing of waste fluids.

A waste fluid storage control device according to the present disclosure includes a waste fluid storage container, a suction device, a sensor, a driving unit, and a computation unit. The waste fluid storage container is configured to store a waste fluid. The suction device communicates with the waste fluid storage container and is configured to generate a negative pressure to draw the waste fluid into the waste fluid storage container. A sensor is configured to measure the pressure. The driving unit is configured to perform driving control upon the suction device in accordance with the measured pressure. The computation unit is configured to detect waste fluid drawing related information from a change in the pressure. The driving unit performs the driving control using an ON period in which the suction device operates and an OFF period in which the suction device stops. The computation unit detects the waste fluid drawing related information using a first change in the pressure in the OFF period or a second change in the pressure in the ON period.

In this configuration in which, for example the OFF period is set, there is a time period in which the pulsation of the suction device does not transmit to the sensor. In the time period in which the pulsation of the suction device does not transmit to the sensor, the pressure measurement accuracy of the sensor enhances. Accordingly, waste fluid drawing related information, such as an air leak, can be accurately acquired.

According to the present disclosure, various states regarding drawing of waste fluids can be accurately detected.

### BRIEF DESCRIPTION OF THE DRAWINGS

FIG. 1 is a diagram illustrating the configuration of a waste fluid storage system according to a first embodiment.

FIG. 2 is a flowchart illustrating a driving control method performed by a driving unit in a waste fluid storage device.

FIG. 3A is a graph representing an exemplary waveform of a driving signal, and FIG. 3B is a graph representing a change in negative pressure over time.

FIGS. 4A and 4B are flowcharts illustrating setting of timing of switching between an ON period and an OFF period in a first specific aspect.

FIGS. 5A and 5B are flowcharts illustrating setting of timing of switching between the ON period and the OFF period in a second specific aspect.

FIG. 6 is a block diagram illustrating an exemplary configuration of a computation unit according to a first embodiment of the present disclosure.

FIGS. 7A and 7B are graphs representing changes in negative pressure over time for description of an air leak detection concept.

FIG. 8 is a graph representing a change in negative pressure over time for description of the concept of detecting the amount of waste fluid.

FIG. 9 is a graph representing a change in negative pressure over time for description of the concept of further suppressing the effect of a pulsation of a pump.

FIGS. 10A and 10B are graphs representing changes in negative pressure over time for description of the concept of detecting a pulsation derived from a living body, and

FIG. 10C is a graph representing a change in negative pressure over time for description of the concept of detecting the blockage of a tube.

FIG. 11A is a graph representing a change in negative pressure over time for description of the concept of detecting a healing state, and FIG. 11B is a graph representing a change in negative pressure over time for description of the concept of detecting the blockage of a tube.

FIG. 12 is a diagram illustrating the configuration of a waste fluid storage system according to a second embodiment.

### DETAILED DESCRIPTION

### First Embodiment

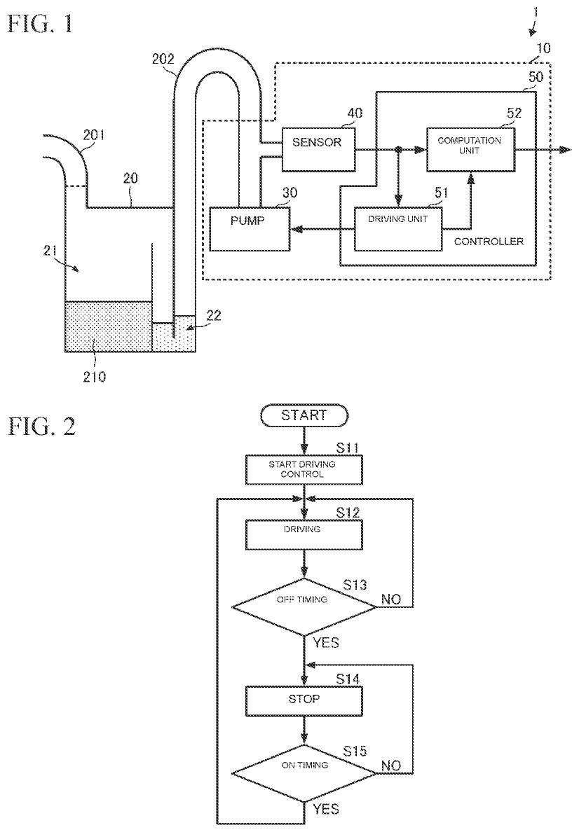

A waste fluid storage control system including a waste fluid storage control device according to the first embodiment of the present disclosure will be described with reference to the accompanying drawings. FIG. 1 is a diagram illustrating the configuration of a waste fluid storage system according to the first embodiment.

As illustrated in FIG. 1, a waste fluid storage system 1 includes a waste fluid storage control device 10, a waste fluid storage container 20, a tube 201, and a tube 202.

The waste fluid storage container 20 is a box having an inner space. The waste fluid storage container 20 includes a waste fluid storage unit 21 and a water seal unit 22. The waste fluid storage unit 21 and the water seal unit 22 communicate with each other. The waste fluid storage unit 21 communicates with the tube 201. The tube 201 is placed in, for example, the affected area of a patient. This affected area corresponds to a “target part” according to the present disclosure. A target in which the tube 202 is placed is not limited to a patient and may be a living body from which fluids need to be drained. The water seal unit 22 communicates with the tube 202. The tube 202 communicates with a pump 30 in the waste fluid storage control device 10.

The waste fluid storage control device 10 includes the pump 30, a sensor 40, and a controller 50. The pump 30 and the sensor 40 connect to the controller 50. The controller 50 includes a driving unit 51 (e.g., a driver or like driving circuitry) and a computation unit 52 (e.g., a processor or like circuitry). The sensor 40 connects to the driving unit 51 and the computation unit 52. The pump 30 connects to the driving unit 51. The pump 30 corresponds to a “suction device” according to the present disclosure.

The pump 30 communicates with the tube 202 as described above. The pump 30 is, for example, a piezoelectric pump. A driving signal having a predetermined driving frequency is input from the driving unit 51 to the pump 30. The pump 30 operates or stops in accordance with the driving signal. More specifically, the pump 30 operates when the driving voltage of the driving signal is not zero, that is, in an ON period, and stops when the driving voltage of the driving signal is zero, that is, in an OFF period.

During the operation of the pump 30, air is transmitted from the tube 202 to the outside via the pump 30. As a result, the waste fluid storage container 20 communicating with the tube 202 has a negative pressure relative to the outside, more specifically, relative to an affected area, and air is drawn from the tube 201. In response to this drawing of air, waste fluids 210 are drawn into and stored in the waste fluid storage unit 21 in the waste fluid storage container 20.

The sensor 40 is a pressure sensor and communicates with the tube 202. The sensor 40 can measure the pressure (negative pressure) of the waste fluid storage container 20, more specifically, the pressure (negative pressure) of the waste fluid storage unit 21 by measuring the pressure of the tube 202 communicating with the waste fluid storage container 20. The sensor 40 outputs the measured negative pressure to the driving unit 51 and the computation unit 52.

The driving unit 51 generates a driving signal on the basis of a negative pressure or a measured time and outputs the driving signal to the pump 30. More specifically, the driving unit 51 sets the ON period and the OFF period on the basis of a negative pressure or a measured time. In the ON period, the driving signal has a predetermined driving voltage that is not 0 V. In the OFF period, the driving signal has a driving voltage of 0 V. Thus, the above ON and OFF periods of the pump 30 are set. Strictly speaking, there are differences between the ON period of the driving unit 51 and the ON period of the pump 30 and between the OFF period of the driving unit 51 and the OFF period of the pump 30 which are made depending on the propagation time of the driving signal, a speed at which the pump 30 responses to the driving signal, and air conveyed by the pump 30. However, it may be considered that these ON periods are substantially the same and these OFF periods are substantially the same in the scope of the present application. Even in consideration of such errors, the ON periods can be construed to be the same and the OFF periods can be construed to be the same in the present application.

The computation unit 52 detects various states regarding the drawing of waste fluids, such as an air leak to be described below, using the negative pressure measured by the sensor 40. The computation unit 52 outputs a result of the detection to, for example, a display (not illustrated).

(Concrete Driving Control Method)

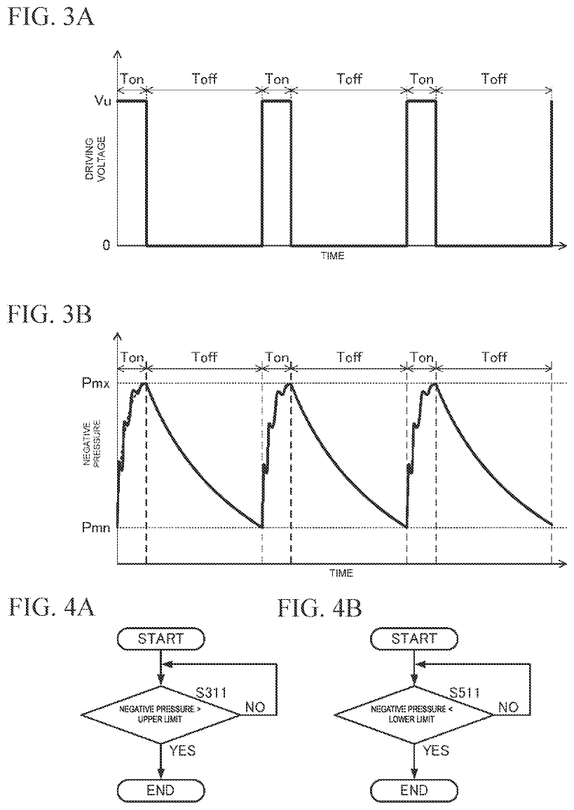

FIG. 2 is a flowchart illustrating a driving control method performed by a driving unit in a waste fluid storage device. FIG. 3A is a graph representing an exemplary waveform of a driving signal, and FIG. 3B is a graph representing a change in negative pressure over time.

As illustrated in FIG. 2, the driving unit 51 starts driving control in response to an operation input for the start of drawing of waste fluids which have been performed upon an operation component, such as a button or a switch, provided on the waste fluid storage control device 10 (S11). When starting the driving control, the driving unit 51 outputs a driving signal of a driving voltage Vu that is not 0 V to the pump 30. The pump 30 is driven in accordance with this deriving signal (S12).

Until an OFF timing (S13: NO), the driving unit 51 continues the driving control (S12). A period in which the driving voltage Vu is applied to the pump 30, that is, a period in which the pump 30 operates, is an ON period Ton (see FIG. 3A).

When the OFF timing comes (S13: YES)), the driving unit 51 stops the driving signal. The driving unit 51 sets the driving voltage to 0 V. The supply of the driving voltage Vu to the pump 30 stops in response to the setting, and the operation of the pump 30 stops (S14). The driving unit 51 continues the stop of supply of the driving voltage Vu until an ON timing (S15: NO). A period in which the supply of the driving voltage Vu to the pump 30 stops, that is, a period in which the pump 30 stops, is an OFF period Toff (see FIG. 3A).

Since the pump 30 stope in the OFF period Toff in this configuration and under this control, the pump 30 does not perform pulsation. Accordingly, the measurement value of a negative pressure, that is, a negative pressure measured by the sensor 40, does not vary because of pulsation. A negative pressure measured by the sensor 40 therefore accurately reflects a negative pressure inside the waste fluid storage container 20. This allows the computation unit 52 to detect information related to the drawing of waste fluids (e.g., an air leak to be described below) using an accurate negative pressure. As a result, information related to the drawing of waste fluids becomes accurate.

The driving voltage Vu in the ON period does not necessarily have to be always constant and may have a time average value. For example, in the case where PWM control is employed, the driving voltage Vu may be substantially constant in the ON period Ton even if an OFF period for the PWM control is set in accordance with a driving frequency having a driving period much shorter than the ON period Ton. That is, the OFF period for the PWM control differs from the OFF period Toff in the present application.

(First Specific Aspect of Setting of OFF Timing and ON Timing)

FIGS. 4A and 4B are flowcharts illustrating setting of timing of switching between the ON period and the OFF period in the first specific aspect. FIG. 4A illustrates the switching from the ON period to the OFF period. FIG. 4B illustrates the switching from the OFF period to the ON period.

For a higher efficiency of drawing waste fluids, the waste fluid storage control device 10 controls driving of a pump to bring a negative pressure inside the waste fluid storage container 20, that is, the negative pressure of the waste fluid storage unit 21, into a predetermined range. In the related art, for example, the driving of a pump is controlled in such a manner that the value (not 0 V) of a driving voltage is adjusted to bring a negative pressure into a predetermined range.

However, the driving unit 51 in the waste fluid storage control device 10 controls the switching between the ON period Ton and the OFF period Toff in accordance with a negative pressure in the first aspect.

For example, the driving unit 51 sets the timing of switching from the ON period Ton to the OFF period Toff on the basis of a relationship between a negative pressure and the upper limit. More specifically, as illustrated in FIG. 4A, the driving unit 51 performs switching from the ON period Ton to the OFF period Toff at a timing when a negative pressure exceeds the upper limit (S311:YES), that is, the absolute value of the negative pressure exceeds the upper limit. On the other hand, the driving unit 51 continues the supply of the driving voltage Vu, that is, the ON period Ton, when the negative pressure is less than or equal to the upper limit in the ON period Ton (S311:NO).

The driving unit 51 sets the timing of switching from the OFF period Toff to the ON period Ton on the basis of a relationship between a negative pressure and the lower limit. More specifically, as illustrated in FIG. 4B, the driving unit 51 performs switching from the OFF period Toff to the ON period Ton at a timing when a negative pressure falls below the lower limit (S511:YES), that is, the absolute value of the negative pressure falls below the lower limit. On the other hand, the driving unit 51 continues the stop of the driving voltage Vu, that is, the OFF period Toff, when the negative pressure is greater than or equal to the lower limit in the OFF period Toff (S511: NO).

Under the above control, the waste fluid storage control device 10 can accurately measure a negative pressure in the OFF period Toff while bringing the negative pressure into the predetermined range. That is, the waste fluid storage control device 10 can achieve both the efficient drawing of waste fluids and the accurate measurement of a negative pressure.

(Second Specific Aspect of Setting of OFF Timing and ON Timing)

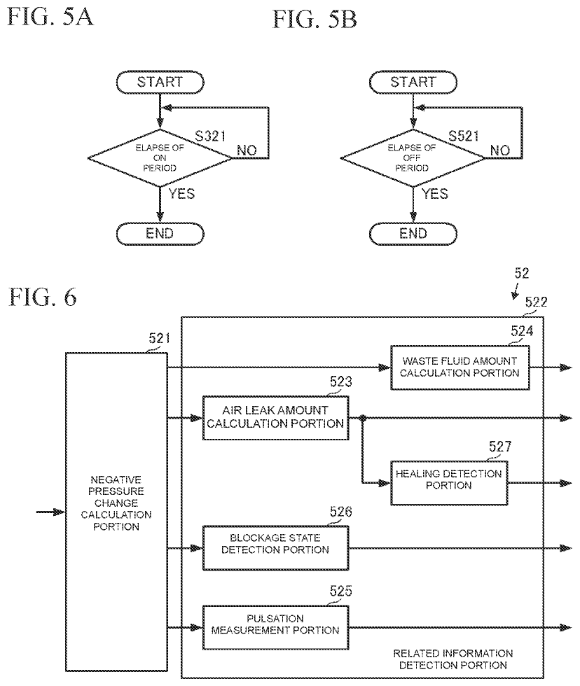

FIGS. 5A and 5B are flowcharts illustrating setting of timing of switching between the ON period and the OFF period in the second specific aspect. FIG. 5A illustrates the switching from the ON period to the OFF period. FIG. 5B illustrates the switching from the OFF period to the ON period.

The driving unit 51 in the waste fluid storage control device 10 controls switching between the ON period Ton and the OFF period Toff on the basis of a time (elapsed time) in the second aspect.

More specifically, as illustrated in FIG. 5A, the driving unit 51 performs time measurement, and performs switching from the ON period Ton to the OFF period Toff at a timing when the ON period Ton has elapsed (S321: YES), that is, when the end time of the ON period Ton has come. On the other hand, the driving unit 51 continues the supply of the driving voltage Vu, that is, the ON period Ton, in the ON period Ton before the end time of the ON period Ton (S321: NO).

As illustrated in FIG. 5B, the driving unit 51 performs time measurement, and performs switching from the OFF period Toff to the ON period Ton at a timing when the OFF period Toff has elapsed (S521: YES), that is, when the end time of the OFF period Toff has come. On the other hand, the driving unit 51 continues the stop of supply of the driving voltage Vu, that is, the OFF period Toff, in the OFF period Toff before the end time of the OFF period Toff (S521: NO).

Under the above control, the switching between the ON period Ton and the OFF period Toff can be performed without necessarily the need to refer to a negative pressure. In the OFF period Toff, a negative pressure can be accurately measured.

In the above aspects, the upper limit of a negative pressure is, for example, approximately −30 cmH₂O (−29 hPa) and the lower limit of a negative pressure is, for example, approximately −2 cmH₂O (−2 hPa). The duration of the ON period Ton is, for example, approximately 0.1 to 20 seconds, and the duration of the OFF period Toff is, for example, approximately 0.1 to 40 seconds.

(Concrete Examples of Configuration of Computation Unit and Information Related to Drawing of Waste Fluids)

FIG. 6 is a block diagram illustrating an exemplary configuration of a computation unit according to the first embodiment of the present disclosure. As illustrated in FIG. 6, the computation unit 52 includes a negative pressure change calculation portion 521 and a related information detection portion 522. The computation unit 52 is formed by, for example, a CPU, an MPU, or an MCU.

The negative pressure change calculation portion 521 calculates the amount of change in negative pressure (the rate of change of a negative pressure with respect to time) and outputs a result of the calculation to the related information detection portion 522.

The related information detection portion 522 detects the following various pieces of information using the amount of change in negative pressure. The related information detection portion 522 functionally includes an air leak amount calculation portion 523, a waste fluid amount calculation portion 524, a pulsation measurement portion 525, a blockage state detection portion 526, and a healing detection portion 527. The related information detection portion 522 may include at least one of them on the basis of related information to be detected.

(Case Where Information Related to Drawing of Waste Fluids Is Air Leak)

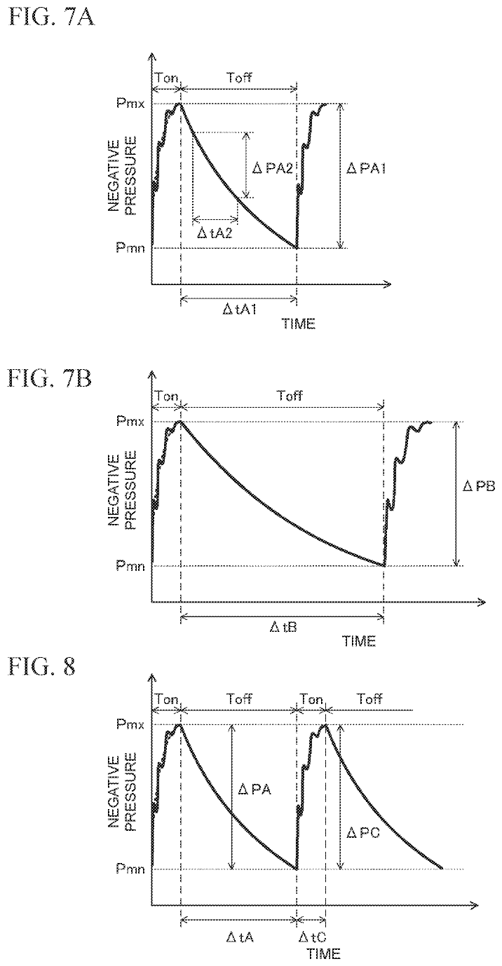

FIGS. 7A and 7B are graphs representing changes in negative pressure over time for description of an air leak detection concept. FIG. 7A illustrates the case where the amount of air leak is large, and FIG. 7B illustrates the case where the amount of air leak is small.

When an air leak occurs, air flows from an affected area into the waste fluid storage container 20 through the tube 201. For example, in the case where the end of the tube 201 is attached to a lung that is an affected area, air leaked from the lung flows from the tube 201 into the inside of the waste fluid storage container 20. The larger the amount of air leak, the faster the decline rate of a negative pressure as illustrated in FIG. 7A. In contrast, the smaller the amount of air leak, the slower the decline rate of a negative pressure as illustrated in FIG. 7B.

That is, there is a predetermined relationship between the amount of air leak and the decline rate of a negative pressure. Using this relationship, the air leak amount calculation portion 523 calculates the amount of change (reduction) in negative pressure per predetermined times, that is, the decline rate of a negative pressure, and calculates the amount of air leak on the basis of the decline rate. The change in negative pressure in the OFF period Toff corresponds to a “first change” in the present disclosure.

In a concrete example, the air leak amount calculation portion 523 calculates a decline rate (ΔPA1/ΔtA1) by dividing a negative pressure change amount ΔPA1 by a time period ΔtA1 of the OFF period Toff in this time period in the case of FIG. 7A. The air leak amount calculation portion 523 calculates the amount of air leak from the decline rate (ΔPA1/ΔtA1). In the case of FIG. 7B, the air leak amount calculation portion 523 calculates a decline rate (ΔPB/ΔtB) by dividing a negative pressure change amount ΔPB by a time period ΔtB in this time period. The air leak amount calculation portion 523 calculates the amount of air leak from the decline rate (ΔPB/ΔtB).

In this method, the time periods ΔtA1 and ΔtB correspond to the time length of the OFF period Toff, and the negative pressure change amounts ΔPA1 and ΔPB correspond to the difference between the upper limit and the lower limit of a negative pressure. However, a time period used for the calculation of a decline rate and the amount of change in negative pressure can be set as appropriate on condition that they are within the OFF period Toff.

For example, the air leak amount calculation portion 523 acquires a negative pressure change amount ΔPA2 in a time period ΔtA2 that is within the OFF period Toff and is shorter than the OFF period Toff as illustrated in FIG. 7A. The air leak amount calculation portion 523 calculates a decline rate (ΔPA2/ΔtA2) from ΔtA2 and ΔPA2. The air leak amount calculation portion 523 calculates the amount of air leak from the decline rate (ΔPA2/ΔtA2).

The air leak amount calculation portion 523 may calculate decline rates in a plurality of periods within the OFF period Toff and calculate the amount of air leak from an average of these decline rates.

In the waste fluid storage control device 10, a negative pressure measured in the OFF period Toff is not affected by the pulsation of the pump 30 and is therefore accurate. Accordingly, the air leak amount calculation portion 523 can accurately calculate the amount of air leak.

(Case Where Information Related to Drawing of Waste Fluids Is the Amount of Waste Fluid)

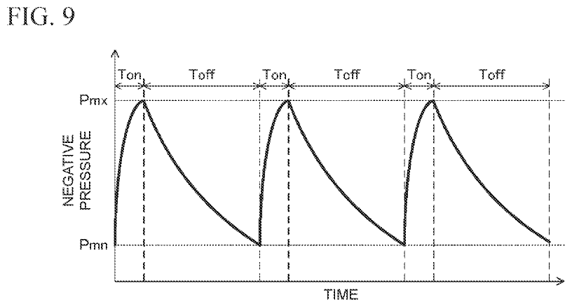

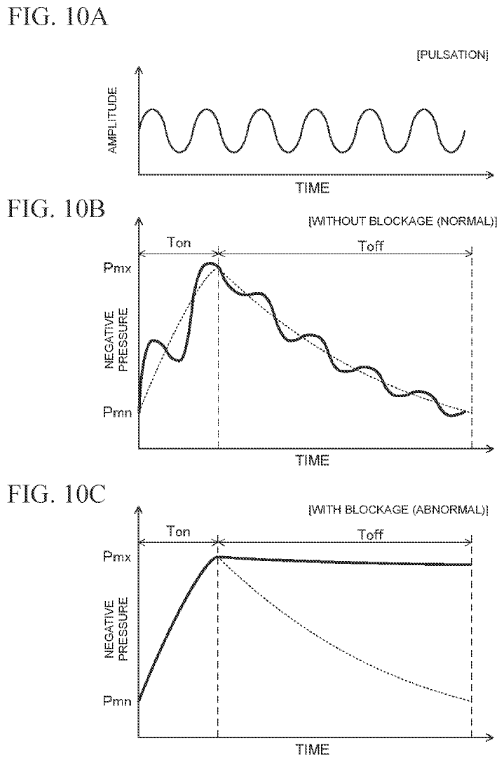

FIG. 8 is a graph representing a change in negative pressure over time for description of the concept of detecting the amount of waste fluid.

The amount of waste fluid stored in the waste fluid storage container 20 and the amount of air leak have influences on a negative pressure increase rate in the ON period Ton and a negative pressure decline rate in the OFF period Toff. Accordingly, the waste fluid amount calculation portion 524 can calculate the amount of waste fluid and the amount of air leak using the negative pressure increase rate and the negative pressure decline rate.

In a concrete example, the waste fluid amount calculation portion 524 calculates an increase rate (ΔPC/ΔtC) by dividing a negative pressure change amount ΔPC in this time period by a time period ΔtC of the ON period Ton as illustrated in FIG. 8. The change in negative pressure in the ON period Ton corresponds to a “second change” in the present disclosure. The waste fluid amount calculation portion 524 calculates a decline rate (ΔPA/ΔtA) by dividing a negative pressure change amount ΔPA in this time period by a time period ΔtA of the OFF period Toff as illustrated in FIG. 8.

The waste fluid amount calculation portion 524 calculates the amount of waste fluid and the amount of air leak using the increase rate (ΔPC/ΔtC) and the decline rate (ΔPA/ΔtA). With this method, the waste fluid amount calculation portion 524 can calculate both the amount of waste fluid and the amount of air leak and also accurately calculate the amount of waste fluid and the amount of air leak by taking both of them into account.

Also in this method, a time period used for the calculation of an increase rate and the amount of change in negative pressure can be set as appropriate on condition that they are within the ON period Ton, and a time period used for the calculation of a decline rate and the amount of change in negative pressure can be set as appropriate on condition that they are within the OFF period Toff.

(Method of Further Reducing Effect of Pulsation of Pump)

FIG. 9 is a graph representing a change in negative pressure over time for description of the concept of further suppressing the effect of a pulsation of a pump.

The pulsation of the pump 30 is suppressed by driving the pump 30 at a high driving frequency. Specifically, the driving frequency is greater than or equal to, for example, approximately 20 kHz. As a result, pulsation that occurs in a negative pressure is suppressed also in the ON period Ton as illustrated in FIG. 9. Accordingly, the amount of change in negative pressure in the ON period Ton can be accurately calculated. For example, the above waste fluid amount calculation portion 524 can more accurately calculate both the amount of waste fluid and the amount of air leak.

(Case Where Information Related to Drawing of Waste Fluids Is Pulsation Derived from Living Body or Blockage of Tube)

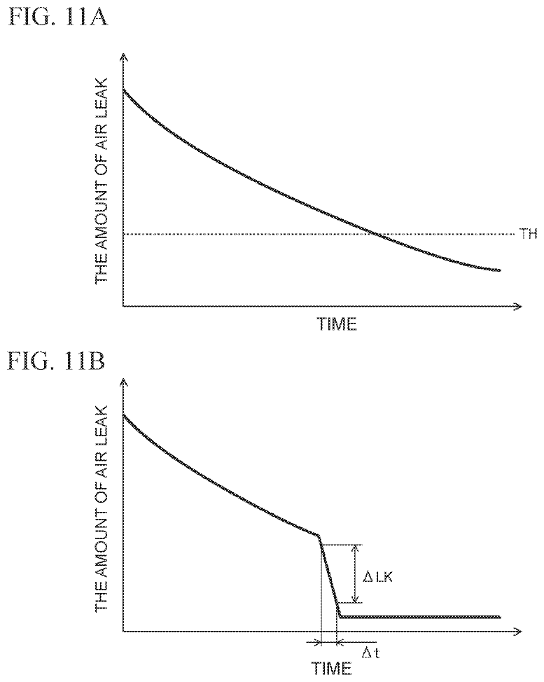

FIGS. 10A and 10B are graphs representing changes in negative pressure over time for description of the concept of detecting a pulsation derived from a living body, and FIG. 10C is a graph representing a change in negative pressure over time for description of the concept of detecting the blockage of a tube.

Pulsation derived from a living body is made by, for example, repeated breathing. This pulsation has a lower frequency and a larger amplitude than the pulsation of the pump 30. Accordingly, in the case where there is the pulsation derived from a living body illustrated in FIG. 10A, the pulsation derived from a living body superimposes on a negative pressure as illustrated in FIG. 10B.

Using this, the pulsation measurement portion 525 detects the periodicity of a negative pressure from, for example, the change in negative pressure over time. When the pulsation measurement portion 525 detects a periodic change in negative pressure, it detects that there is the pulsation of a patient. On the other hand, when the pulsation measurement portion 525 does not detect a periodic change in negative pressure, it detects that there is no pulsation of a patient.

At that time, the pulsation measurement portion 525 can more accurately detect the presence or absence of a pulsation by detecting that a smoothed negative pressure has gradually increased in the ON period Ton and detecting that a smoothed negative pressure has gradually decreased in the OFF period Toff. The pulsation measurement portion 525 can also measure pulsation by calculating the period of a negative pressure by, for example, the Fourier computation.

In the case where the blockage of the tube 201 occurs, a negative pressure shows little change in the OFF period Toff as illustrated in FIG. 10C. Accordingly, the blockage state detection portion 526 can detect that the tube 201 is in the blockage state when there is little change in negative pressure in the OFF period Toff.

(Case Where Information Related to Drawing of Waste Fluids Is Healing State or Blockage of Tube)

FIG. 11A is a graph representing a change in negative pressure over time for description of the concept of detecting a healing state, and FIG. 11B is a graph representing a change in negative pressure over time for description of the concept of detecting the blockage of a tube.

When the healing of an affected area progresses, the amount of air leak decreases. Accordingly, the amount of air leak gradually decreases depending on the progress state of healing of an affected area as illustrated in FIG. 11A. The healing detection portion 527 detects a healing state from the change in the amount of air leak over time. For example, a healing detection threshold TH is set for the amount of air leak as illustrated in FIG. 11A. When the healing detection portion 527 detects that the amount of air leak is less than or equal to the healing detection threshold TH, it detects that healing has progressed to a certain degree. The healing detection portion 527 may detect the state of healing using the amount of change in air leak from the initial amount of air leak. For example, the healing detection portion 527 can detect that the degree of progress of healing is higher, that is, the degree of improvement of an affected area is higher when the amount of change in air leak from the initial amount of air leak is larger.

Such change in the amount of air leak can also be used for the detection of the blockage of a tube. The amount of air leak gradually decreases depending on the progress state of healing as illustrated in FIG. 11A. That is, in general, the rapid decrease in the amount of air leak illustrated in FIG. 11B rarely occurs.

Using this, the healing detection portion 527 calculates the rate of change of the amount of air leak with respect to time, that is, calculates a value (ΔLK/Δt) by dividing the amount of change in air leak ΔLK in a predetermined time period Δt by the predetermined time period Δt. The healing detection portion 527 sets a threshold value for the rate of change of the amount of air leak with respect to time. When the rate of change of the amount of air leak with respect to time exceeds the threshold value, the healing detection portion 527 detects the blockage of a tube. Using such a method, the blockage of a tube can be detected.

### Second Embodiment

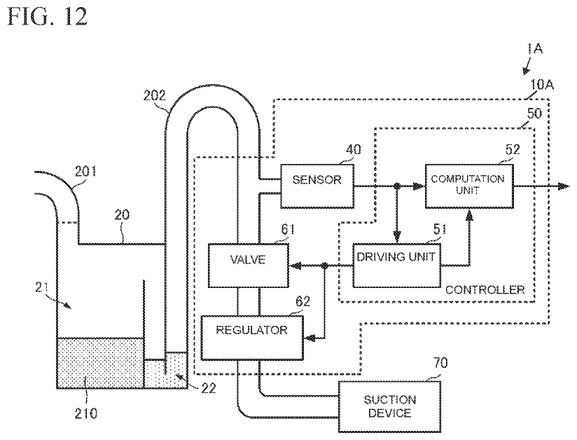

A waste fluid storage control system including a waste fluid storage control device according to the second embodiment of the present disclosure will be described with reference to a drawing. FIG. 12 is a diagram illustrating the configuration of a waste fluid storage system according to the second embodiment.

As illustrated in FIG. 12, a waste fluid storage system 1A according to the second embodiment differs from the waste fluid storage system 1 according to the first embodiment in the configuration of a waste fluid storage control device 10A. The other configuration of the waste fluid storage system 1A is the same as that of the waste fluid storage system 1, and the description of the same parts will be omitted.

The waste fluid storage control device 10A differs from the waste fluid storage control device 10 in that it includes a valve 61 and a regulator 62 instead of the pump 30. The other configuration of the waste fluid storage control device 10A is the same as that of the waste fluid storage control device 10, and the description of the same parts will be omitted.

The valve 61 communicates with the tube 202. The valve 61 communicates with the suction device 70 that is an external device via the regulator 62. In this configuration, the valve 61 and the regulator 62 correspond to the “suction device” according to the present disclosure.

A period in which the valve 61 is controlled to be in an open state is the ON period Ton, and a period in which the valve 61 is controlled to be in a closed state is the OFF period Toff. The regulator 62 controls the suction pressure of the suction device 70. For example, the regulator 62 controls a suction pressure to stabilize a negative pressure generated in the waste fluid storage container 20. The regulator 62 does not necessarily have to be provided.

Even with this configuration, the waste fluid storage control device 10A can obtain an operational effect similar to that of the above-described waste fluid storage control device 10.

Configurations according to the above embodiments can be combined as appropriate.

### REFERENCE SIGNS LIST

1 and 1A waste fluid storage system
10 and 10A waste fluid storage control device
20 waste fluid storage container
21 waste fluid storage unit
22 water seal unit
30 pump
40 sensor
50 controller
51 driving unit
52 computation unit
61 valve
62 regulator
70 suction device
201 and 202 tube
210 waste fluids
521 negative pressure change calculation portion
522 related information detection portion
523 air leak amount calculation portion
524 waste fluid amount calculation portion
525 pulsation measurement portion
526 blockage state detection portion
527 healing detection portion

1. A waste fluid storage control device comprising:
a suction device configured to communicate with a waste fluid storage container that stores a waste fluid, the suction device further configured to generate a negative pressure to draw the waste fluid into the waste fluid storage container;
a sensor configured to measure the negative pressure;
a driver configured to drive the suction device in accordance with the measured negative pressure; and
a processor configured to detect waste fluid drawing related information from a change in the negative pressure,
wherein the driver is further configured to drive the suction device during an ON period in which the suction device operates and an OFF period in which the suction device stops, and
wherein the processor is further configured to detect the waste fluid drawing related information based on a first change in the negative pressure in the OFF period or a second change in the negative pressure in the ON period.

2. The waste fluid storage control device according to claim 1,
wherein the waste fluid drawing related information indicates an air leak in the waste fluid storage container, and
wherein the processor is further configured to detect the air leak in the waste fluid storage container based on a rate of the first change.

3. The waste fluid storage control device according to claim 2,
wherein the waste fluid drawing related information further indicates a state of a waste fluid drawing target part or a blockage of a tube into which the waste fluid is drawn, and
wherein the processor is further configured to detect the state of the waste fluid drawing target part or the blockage of the tube into which the waste fluid is drawn based on a change in the air leak over time.

4. The waste fluid storage control device according to claim 1,
wherein the waste fluid drawing related information further indicates an amount of the waste fluid in the waste fluid storage container, and
wherein the processor is further configured to detect the amount of the waste fluid in the waste fluid storage container based on a rate of the first change and a rate of the second change.

5. The waste fluid storage control device according to claim 1,
wherein the waste fluid drawing related information further indicates a pulsation of a target living body for which drawing of the waste fluid is performed, and
wherein the processor is further configured to detect the pulsation based on a periodic change included in the first change or the second change.

6. The waste fluid storage control device according to claim 1,
wherein the waste fluid drawing related information further indicates a blockage of a tube into which the waste fluid is drawn, and
wherein the processor is further configured to detect the blockage of the tube based on the first change.

7. The waste fluid storage control device according to claim 1,
wherein the driver is further configured to store an upper limit and a lower limit of an absolute value of the negative pressure,
wherein the driver is further configured to switch drive of the suction device such from the ON period to the OFF period when the absolute value of the negative pressure exceeds the upper limit, and
wherein the driver is further configured to switch drive of the suction device from the OFF period to the ON period when the absolute value of the negative pressure falls below the lower limit.

8. The waste fluid storage control device according to claim 1, wherein the driver is further configured to drive the suction device at a driving frequency greater than or equal to approximately 20 kHz.

9. The waste fluid storage control device according to claim 2,
wherein the waste fluid drawing related information further indicates an amount of the waste fluid in the waste fluid storage container, and
wherein the processor is further configured to detect the amount of the waste fluid in the waste fluid storage container based on a rate of the first change and a rate of the second change.

10. The waste fluid storage control device according to claim 3,
wherein the waste fluid drawing related information further indicates an amount of the waste fluid in the waste fluid storage container, and
wherein the processor is further configured to detect the amount of the waste fluid in the waste fluid storage container based on a rate of the first change and a rate of the second change.

11. The waste fluid storage control device according to claim 2,
wherein the waste fluid drawing related information further indicates a pulsation of a target living body for which drawing of the waste fluid is performed, and
wherein the processor is further configured to detect the pulsation based on a periodic change included in the first change or the second change.

12. The waste fluid storage control device according to claim 3,
wherein the waste fluid drawing related information further indicates a pulsation of a target living body for which drawing of the waste fluid is performed, and
wherein the processor is further configured to detect the pulsation based on a periodic change included in the first change or the second change.

13. The waste fluid storage control device according to claim 4,
wherein the waste fluid drawing related information further indicates a pulsation of a target living body for which drawing of the waste fluid is performed, and
wherein the processor is further configured to detect the pulsation based on a periodic change included in the first change or the second change.

14. The waste fluid storage control device according to claim 2,
wherein the waste fluid drawing related information further indicates a blockage of a tube into which the waste fluid is drawn, and
wherein the processor is further configured to detect the blockage of the tube based on the first change.

15. The waste fluid storage control device according to claim 3,
wherein the waste fluid drawing related information further indicates a blockage of a tube into which the waste fluid is drawn, and
wherein the processor is further configured to detect the blockage of the tube based on the first change.

16. The waste fluid storage control device according to claim 4,
wherein the waste fluid drawing related information further indicates a blockage of a tube into which the waste fluid is drawn, and
wherein the processor is further configured to detect the blockage of the tube based on the first change.

17. The waste fluid storage control device according to claim 5,
wherein the waste fluid drawing related information further indicates a blockage of a tube into which the waste fluid is drawn, and
wherein the processor is further configured to detect the blockage of the tube based on the first change.

18. The waste fluid storage control device according to claim 2,
wherein the driver is further configured to store an upper limit and a lower limit of an absolute value of the negative pressure,
wherein the driver is further configured to switch drive of the suction device such from the ON period to the OFF period when the absolute value of the negative pressure exceeds the upper limit, and
wherein the driver is further configured to switch drive of the suction device from the OFF period to the ON period when the absolute value of the negative pressure falls below the lower limit.

19. The waste fluid storage control device according to claim 2, wherein the driver is further configured to drive the suction device at a driving frequency greater than or equal to approximately 20 kHz.
